(19) United States
(12) Patent Application Publication
(10) Pub. No.: US 2003/0064965 A1
(43) Pub. Date: Apr. 3, 2003

(54) Method of delivering drugs to a tissue using drug-coated medical devices

(76) Inventor: Jacob Richter, Ramat Hasharon (IL)

Correspondence Address: MORGAN & FINNEGAN, L.L.P., 345 Park Avenue, New York, NY, 10154-0053, US

(21) Appl. No.: 10/076,723

(22) Filed: Feb. 14, 2002

Related U.S. Application Data
(60) Provisional application No. 60/326,571, filed on Oct. 2, 2001.

(51) Int. Cl. A61K 31/675; A61K 31/66; A61K 9/127

(52) U.S. Cl. 514/102; 514/89; 604/19

(57) ABSTRACT

The present invention relates to a method of delivering drugs having anti-proliferative activity in the cardiovascular system to a tissue or circulation using a drug-coated medical device. The drug-coated medical device is brought into contact with the target tissue or circulation and the drugs are quickly released into the area surrounding the device in a short time after the contact step. The release times may include 30 seconds, 1 minute or 3 minutes. Once the therapeutic drugs are released, they are quickly and effectively absorbed by the surrounding cells or circulation. The therapeutic drug may have sustained anti-proliferative activity and thus a prolonged effect. The therapeutic drug, which inhibits proliferative activity in the cardiovascular system, may be preferably encapsulated in a controlled release carrier. In a preferred embodiment, the controlled release carrier may be a liposome, drug aggregate, microparticle or nanoparticle and the therapeutic agent may be a bisphosphonate.

### FIELD OF THE INVENTION

The present invention relates to the field of medicinal devices and their use in delivering drugs to a particular tissue or body lumen.

### BACKGROUND OF THE INVENTION

Various methods are presently known in the art for the delivery of a pharmaceutical composition for the treatment of various medical conditions. The pharmaceutical composition may be provided to a human or veterinary patient in need of therapeutic treatment, by a variety of routes, such as, for example, subcutaneous, topical, oral, intraperitoneal, intradermal, intravenous, intranasal, rectal, intramuscular, and within the pleural cavity. Administration of pharmaceutical compositions is usually accomplished orally or parenterally.

However, it has become increasingly common to treat a variety of medical conditions by introducing an implantable medical device partly or completely into the esophagus, trachea, colon, biliary tract, urinary tract, vascular system or other location within a human or veterinary patient. For example, many treatments of the vascular system entail the introduction of a device such as a stent, a catheter, a balloon, a guide wire, a cannula or the like.

Exposure, however, to a medical device which is implanted or inserted into the body of a patient can cause the body tissue to exhibit adverse physiological reactions. For instance, the insertion or implantation of certain catheters or stents can lead to the formation of emboli or clots in blood vessels. Similarly, the implantation of urinary catheters can cause infections, particularly in the urinary tract. Other adverse reactions to implanted or temporary treatment medical devices whether introduced by an operation or by a minimally invasive technique, include cell proliferation which can lead to hyperplasia, occlusion of blood vessels, platelet aggregation, rejection of artificial organs, calcification, and impairment of device function.

For example, when a medical device is introduced into and manipulated through the vascular system, the blood vessel walls can be disturbed or injured. Clot formation or thrombosis, and/or cell proliferation often results at the injured site, causing stenosis (i.e., closure) of the blood vessel. Additionally, if the medical device is left within the patient for an extended period of time, thrombus may form on the device itself with subsequent cell proliferation, again causing stenosis. As a result, the patient is placed at risk of a variety of complications, including heart attack or other ischemic disease, pulmonary embolism, and stroke. Thus, the use of such a medical device can entail the risk of precisely the problems that its use was intended to ameliorate. Also, the intended function of such a medical device can be impaired.

A further method in which blood vessels undergo stenosis is through disease. Probably the most common disease causing stenosis of blood vessels is atherosclerosis. Atherosclerosis is a condition which commonly affects arteries, including, for example, the coronary arteries, the aorta, the iliofemoral arteries, and the carotid arteries. Obstruction of an artery or arteries is caused by atherosclerotic plaques of lipids, fibroblasts and other cells, and fibrin proliferate. As the obstruction increases, a critical level of stenosis is reached, to the point where the flow of blood past the obstruction is insufficient to meet the metabolic needs of the tissue downstream of the obstruction. The result is ischemia. Atherosclerosis is the most common form of vascular disease and leads to insufficient blood supply to body organs, which can result in heart attacks, strokes, kidney failure, and impairment of other ischemic organs. Atherosclerosis is a form of vascular injury in which the vascular smooth cells in the artery wall undergo hyperproliferation and invade and spread into the inner vessel lining, which can make the vessels susceptible to complete blockage when local blood clotting occurs. Such blockage can lead to death of the tissue served by that artery. In the case of a coronary artery, this blockage can lead to myocardial infarction and death.

However, many medical devices and therapeutic methods are known to those skilled in the art for the treatment of atherosclerotic disease. One particularly useful therapy for certain atherosclerotic lesions is percutaneous transluminal angioplasty (PTA). During PTA, a balloon-tipped catheter is inserted in a patient's artery, wherein the balloon is deflated. The tip of the catheter is advanced to the site of the atherosclerotic plaque to be dilated. The balloon is placed within or across the stenotic segment of the artery, and then inflated. Inflation of the balloon “cracks” the atherosclerotic plaque and expands the vessel, thereby relieving the stenosis, at least in part.

Furthermore, atherosclerosis or coronary artery blockage can be treated with coronary artery bypass surgery and/or with a stent. The medical devices and therapeutic methods described supra, may initially appear to be successful, but are in effect sometimes undone by the effect of restenosis, the recurrence of stenosis, after such a treatment.

Restenosis is the formation of new blockages at the site of the angioplasty or stent placement or the anastomosis of the bypass. There are two major mechanisms for restenosis. The first is by thrombosis, or blood clotting, at the site of treatment. The risk of thrombosis is the greatest immediately after angioplasty, because the resultant tissue trauma tends to trigger blood clotting. This form of restenosis is greatly reduced by using anti-clotting drugs both during and after the procedure.

The second form of restenosis is tissue growth at the site of treatment. This form of restenosis, a hyperproliferation of the vascular smooth muscle cells that forms a layer in the wall of a blood vessel, tends to occur during the first three to six months after the procedure, and is not prevented by anti-clotting drugs. This form of restenosis can be thought of as resulting from “over exuberant” tissue healing and regeneration after the trauma of angioplasty and/or stent placement.

To reduce adverse effects caused by implanted medical devices, such as restenosis, pharmaceuticals, such as anticoagulants and antiproliferation drugs, have been administered in or on medical devices. These methods need to release their active ingredients slowly. Indeed, prior art therapeutic methods include slow controlled release, over a predetermined time, of the therapeutic agent coated upon a permanent implant device to the nearby tissue.

Various methods of adjunctive therapy for fighting the restenosis component generated by smooth muscle cell (“SMC”) proliferation have been proposed and evaluated; with the leading ones, and their respective status, listed below.

Anti-platelet agents

The use of anti-platelet or anti-thrombotic agents such as Heparin during and after the therapeutic procedure (e.g. angioplasty and/or stent placement) was expected to inhibit or at least decrease SMC proliferation due to the prevention of PDGF (Platelet Derived Growth Factor) release. However, this approach had no significant effect on SMC proliferation. The anti-platelet or anti-thrombotic agents were administered either orally, intravenously or via a coated implantable medical device.

Radioactive stent

The method and procedure of the ISOstent includes bombarding SST stents with ³²P, implanting the radioactive stent, and preventing SMC proliferation by β radiation from the stent. The ISOstent was initially viewed as successful, but was subsequently abandoned upon the realization that the radiation effect was uncontrollable at the edges of the stent. Additionally, at various points away from the stent the radiation increased restenosis, a phenomenon that is referred to as the “candy-wrapper” effect. This edge effect lesion, which takes on the appearance of a bar bell or a “candy-wrapper” when visualized with an angiogram, is itself a form of restenosis, and represents a significant and difficult-to-treat result. An additional contributing factor to the ineffectiveness of the radioactive stent may have been the limited shelf-life of the ³²P isotope and its half-life time of 14 days.

Radiation therapy

Brachitherapy, an additional adjunctive therapy for the treatment of SMC proliferation, is the use of radiation in coronary arteries to prevent cell proliferation and tissue growth.

Intra-coronary radiation is administered during a special heart catherization procedure. The radiation itself is delivered by a special catheter designed to apply radiation to a localized area. The catheter is passed through the coronary arteries, and to the target area where the radiation is then administered. Two varieties of radiation have been used thus far: gamma radiation and beta radiation.

However, there has been a steady decline in the use of radiation therapy for the treatment of SMC proliferation. This decline can be attributed to the adverse effects radiation therapy has had on cell types other than the SMC, and the cost of using the therapy due to the complex procedures and special equipment involved. In addition to these documented problems with brachitherapy, other potential problems are also possible. For example, radiation may weaken the walls of the coronary artery, and produce an aneurysm, a ballooning out of the arterial wall, which is a potentially hazardous condition.

The main reason, however, for the decline in popularity of radiation therapy as a method of adjunctive therapy for the treatment of SMC proliferation, is the development and clinical trial phase of drug-coated stents.

Drug-embedded stents

The most promising adjunctive therapy known in the art is using stents coated with polymers that either degrade or slowly release the encapsulated drug, thereby generating an effective concentration of the drug over a predetermined time period, which successfully inhibits or reduces SMC proliferation. Typical drugs used to coat the stents are toxins that interfere in different stages of cell division thereby inhibiting SMC proliferation. These toxins include, but are not limited to, Rapamycine and Taxol. While the exact therapeutic window in humans is unknown, the one in animals, specifically pigs, is about 1:5. This very narrow therapeutic window may provide a serious limitation when more complex lesions are treated and when stents with non-uniform strut density are used, resulting in non-uniform drug distribution.

Gene therapy

Additionally, a new technique, gene therapy, has been developed to coat stents with a polymer that can deliver DNA to the local tissue. While it is postulated that local gene therapy will limit SMC proliferation, thus inhibiting restenosis without any significant adverse effects, this therapy still requires further testing and lengthy clinical trials.

Indeed, while various techniques are presently practiced in the prior art for such localized delivery of a therapeutic agent from a drug coated medical device, the presence of the therapeutic agent is often transient. The agent is typically washed away by moving fluids within the body, or quickly neutralized by the biochemical process. On the other hand, the therapeutic agent may be covered by a porous polymer layer which does not quickly release the agent for immediate and effective use. Typically, the prior art therapeutic agents slowly absorb into surrounding tissue or circulation and thus require controlled time-release carriers which allow for relatively slow, controlled diffusion of the therapeutic agent out of the carrier. If therapeutic agents which require a slow diffusion into the surrounding tissue or cell in order to be effective are quickly released into the surrounding tissue or circulation, their presence is often transient, and they are typically washed away, since there is insufficient time for the agents to effectively diffuse into the surrounding tissue.

Thus, it would be desirable to develop devices and methods for reliably delivering therapeutic agents, drugs, or bioactive materials directly into a localized tissue area during or following a medical procedure, so as to treat or prevent conditions and diseases. Indeed, the device should quickly release the therapeutic agent in an effective and efficient manner and the therapeutic agent shall immediately absorb into the tissue or circulation. Additionally, it may also be desirable for the therapeutic agent or drug to have sustained anti-proliferative activity despite its immediate release into the tissues or circulation.

### SUMMARY OF THE INVENTION

The present invention relates to a method of delivering drugs having activity, such as anti-proliferative activity in the cardiovascular system, to tissues in the body or within a circulation in conjunction with a device treatment. These drugs are coated onto a medical device and are released from the device in a short time, preferably less than three minutes, after their exposure to a tissue or circulation. Methods of releasing the drug include activating a trigger mechanism, or having the physiological conditions in the body trigger the release. The method of the present invention comprises contacting the tissue or circulation with a device which is coated with a therapeutic drug, wherein the drug is released into the circulation or the tissues surrounding the device in a short time after the contacting or immediately by the activation of a trigger mechanism (either actively or by the physiological conditions). The therapeutic drug is then quickly, effectively and efficiently absorbed or taken into the tissue, cells or circulation. The therapeutic drugs for coating the device include but are not limited to medicines, proteins, adjuvants, lipids and other compounds which ameliorate the tissue or circulation surrounding the device. Additionally, the drug may be encapsulated in particles or controlled release carriers including liposomes, microparticles, and nanoparticles, which are coated upon the device, or bonded to it. Alternatively, the drug may be an aggregate or flocculate of the drug or drug formulation. These drug aggregates are considered a type of particle, as described herein. The therapeutic drug or drug formulation may have sustained anti-proliferative activity and thus a prolonged effect. One example of a group of drugs useful in the present invention to inhibit proliferative activity in the cardiovascular system, specifically smooth muscle cell proliferation, are bisphosphonates (BP).

In a further embodiment, the present invention relates to a medical device which has a layer of therapeutic drug, having anti-proliferative activity in the cardiovascular system, applied to its exterior. The medical device is contacted with a tissue or circulation such that the drug is released from the medial device and into the surrounding tissue or circulation in less than 5 minutes after contact.

In yet a further embodiment, the present invention relates to a medical device which has a plurality of particles dispersed on its surface, each particle encapsulating a therapeutic drug or a combination of therapeutic drugs having anti-proliferative activity in the cardiovascular system. The particles may preferably be liposomes, microparticles or nanoparticles. The medical device is contacted with a tissue or circulation such that the drug is released from the particle and into the surrounding tissue or circulation in less than 5 minutes after contact.

The method of the invention allows the release of drugs and drug formulations from a device that is not permanently implanted in the body, due to its immediate release. However, the method of the invention is equally applicable and effective, as released from a permanently implanted device.

### DETAILED DESCRIPTION OF THE INVENTION

The present invention relates to a method of delivering drugs in a target-specific manner, through the use of drug-coated medical devices. The claimed method provides a therapy that targets the traumatized area by geometric proximity or in combination with a systemic effect (i.e. delivery from a medical device, such as, for example, a balloon catheter or other device). The drug of the present invention provides anti-proliferative therapeutic activity to the cardiovascular system. The drug is effective while being released into the tissue or surrounding circulation. The drug of the present invention does not require a delayed or long term release and essentially activates anti-proliferative activity immediately upon contact with the cells of the target tissue or circulation. The drug may have sustained anti-proliferative activity and thus, a prolonged effect. The drug is preferably released in less than about three minutes from the time of its initial contact with the tissue or circulation. One method of releasing the drug includes activating a trigger mechanism or having one activated by physiological conditions. For example, the drug may be released within seconds, such as 30 seconds, 40 seconds, 50 seconds, or within minutes such as 1 minute, 2 minutes and up to 3 minutes.

The drugs coated upon the medical devices and thus useful in the present invention are delivered to the target tissue in a short time after the device's initial contact with the targeted tissue or surrounding circulation, i.e., there is a relatively quick release of the drug from the medical device to the tissue. The drugs which can be used in the present invention provide anti-proliferative activity in the cardiovascular system.

In one embodiment, the activity of the drug may be sustained and the drug exhibits a prolonged anti-proliferative effect. Therefore, the drug does not require a delayed or prolonged release and as such, the release can be immediate. Accordingly, the drug may be attached to a device that is not a permanent implant but rather briefly contacts the tissue or circulation such as a balloon catheter. However, the drug may also be released from a permanent implant. Additionally, due to its sustained effect, the drug may also be encapsulated in a particle which may enhance its uptake by the target tissue or cells. Thus, the particles, which provide an effective uptake of the therapeutic agent, may not only be coated on devices which briefly contact the tissue or circulation, such as a balloon catheter, but, may also be coated on a permanent device such as a stent. Thus, the particles coated on the permanent device will be quickly released into the surrounding tissue or circulation.

The drugs may be directly applied to the medical device, may be applied in a composite, wherein the drugs are mixed with other reagents, or may be encapsulated within drug release particles such as liposomes, microparticles, nanoparticles, or aggregates of the drug. The particles may include inert polymeric particles, such as, for example, microparticles or nanoparticles. Alternatively, the particles may comprise biologically derived reagents, such as, for example, lipids, sugars, carbohydrates, proteins and the like. Specifically, such particles are release carriers which provide an effective release of the therapeutic agent to the target tissue or cells. The therapeutic agent formulation may be specifically taken up by cells of the white blood-cell lineage, such as macrophages or monocytes. By this means, the drugs are delivered in a target-specific manner, without the need to provide a full dosage of drugs to the entire body through conventional drug delivery routes as discussed above. Indeed, providing the therapeutic agent in a localized manner or to specific cells can avoid the undesired side effects of such large doses. The drug release carriers are preferably biodegradable, so that when they are brought into contact with the target tissue or circulation or when taken into specific cells, the drug or therapeutic agent is quickly released from the carrier, and then the biodegradable carrier is itself, in due time, removed by natural body processes.

In one embodiment of the present invention the particles or release carriers include, but are not limited to, semi-synthetic polyacryl starch microparticles, other biodegradable microparticles containing the therapeutic agent, ethyl cellulose, poly-L-lactic acid, heptakis (2,6-di-O-ethyl)-beta-cyclodextrin, polyalkylcyanoacrylate nanocapsules, polymethylacrylate, monocarboxycellulose, alginic acid, hyaluronic acid, lipid bilayer beads, polyvinylpyrollidone, polyvinyl alcohol, albumin, lipid carriers of continuous phase (non-microparticle type), nanoparticles, and known agents by those skilled in the art for the release of therapeutic agents. Nanoparticles are preferably spherical or non-spherical polymeric particles that are 30-500 nm in diameter.

In a further embodiment of the present invention, the therapeutic agent or drug may be encapsulated within, or form itself, a liposome, colloid, aggregate, particle, flocculate or other such structure known in the art for encapsulation of drugs. The encapsulation material itself may have a known and predetermined rate of biodegradation or bioerosion, such that the rate of release and amount released is a function of the rate of biodegradation or bioerosion of the encapsulation material. Preferably, the encapsulation material should provide a relatively quick release rate.

In yet a further embodiment of the present invention, the particles, or release carriers, may be supported within the matrix of a macrostructure. Particles or controlled release carriers, as previously discussed, include, but are not limited to microparticles, nanoparticles, colloids, aggregates, liposomes, particles, or flocculates. Materials used to provide the macrostructure include, but are not limited to, fibrin gels, hydrogels, or glucose. Non-limiting examples of particles supported within a macrostructure include a fibrin gel with colloid suspended within it; a hydrogel with liposomes suspended within it; a polymeric macrostructure with macroaggregated albumin suspended within it; glucose with liposomes suspended within it; or any of the foregoing further including liposomes, flocculants microparticles, nanoparticles, or other particles containing or having dispersed therein a drug or therapeutic agent. In the use of this invention it need not be that the macrostructures nor the particles be entirely bioabsorbed. For example if fibrin or collagen is used to provide the macrostructure, such materials are biodegradable yet can persist in the extracellular matrix for substantial lengths of time.

In one embodiment of the invention, the drug or therapeutic agent is encapsulated within liposomes. Liposomes may be submicroscopic, i.e., preferably greater than 100 nm in size, capsules consisting of a double membrane containing various lipids. One such lipid is a phospholipid, a natural material commonly isolated from soy beans. Liposomes are nontoxic and generally recognized as safe by the FDA. Liposomes can be characterized as a hollow flexible sphere containing an aqueous internal compartment surrounded by an external aqueous compartment. Any material trapped inside the liposome is protected from the external aqueous environment. The lipid bilayer acts as a barrier and limits exchange of materials inside, with materials outside the membrane. Furthermore, the lipid bilayers are hydrophobic and can “entrap” and retain similar types of substances. The rate of release of an encapsulated therapeutic agent or drug from a liposome can be, for example, controlled by varying the fatty acid composition of the phospholipid acyl groups, or by providing elements which are embedded in the lipid bilayers, which specifically allow a controlled and rapid release of the encapsulated drug from the liposomes. In practice, chemical modification of the phospholipid acyl groups is accomplished by either chemically modifying the naturally derived materials, or by selecting the appropriate synthetic phospholipid. The embedded elements in the liposome may be biologically- or bioengineering-derived proteins, polypeptides or other macromolecules to selectively provide pores in the liposome wall.

Liposomes are highly advanced assemblages consisting of concentric closed membranes formed by water-insoluble polar lipids. The lipids comprising the membrane may be selected from the group consisting of natural or synthetic phospholipids, mono-, di-, or triacylglycerols, cardiolipin, phosphatidylglycerol, phosphatidic acid, or analogues thereof. Preferably, the liposome formulations are prepared from a mixture of various lipids.

The natural phospholipids are typically those from animal and plant sources, such as phosphatidylcholine, phosphatidylethanolamine, sphingomyelin, phosphatidylserine, or phosphatidylinositol. Synthetic phospholipids typically are those having identical fatty acid groups, including, but not limited to, dimyristoylphosphatidylcholine, dioleoylphosphatidylcholine, dipalmitoylphosphatidylcholine, distearoylphosphatidylcholine and the corresponding synthetic phosphatidylethanolamines and phosphatidylglycerols.

Other additives such as cholesterol, glycolipids, fatty acids, sphingolipids, prostaglandins, gangliosides, neobee, niosomes, or any other natural or synthetic amphophiles can also be used in liposome formulations, as is conventionally known for the preparation of liposomes.

Stability, rigidity, and permeability of the liposomes are altered by changes in the lipid composition. Membrane fluidity is generally controlled by the composition of the fatty acyl chains of the lipid molecules. The fatty acyl chains can exist in an ordered, rigid state or in a relatively disordered fluid state. Factors affecting rigidity include chain length and degree of saturation of the fatty acyl chains and temperature. Larger chains interact more strongly with each other so fluidity is greater with shorter chains. Saturated chains are more flexible than unsaturated chains. Transition of the membrane from the rigid to the fluid state occurs as the temperature is raised above the “melting temperature”. The melting temperature is a function of the length and degree of unsaturation of the fatty acyl chain. In one embodiment, the liposomes, drug aggregates, microparticles, or nanoparticles are created in a pre-selected size that are preferably taken up by macrophages and monocytes. Thus, the liposomes act within the macrophages to incapacitate them or to inhibit their activity. In a preferred embodiment of the present invention, the liposomes are greater than 100 nm.

In addition to temperature and lipid composition, inclusion of a sterol, such as cholesterol, or a charged amphiphile can alter the stability, rigidity and permeability of the liposome by altering the charge on the surface of the liposome and increasing the distance between the lipid bilayers. Proteins and carbohydrates may be incorporated into the liposomes to further modify their properties. (See U.S. Pat. No. 4,921,757 entitled “System for Delayed and Pulsed Release of Biologically Active Substances,” issued May 11, 1990).

The therapeutic agent either directly coated upon or encapsulated and suspended upon a medical device shall be quickly released into the surrounding tissue or circulation of the cardiovascular system once the medical device has been implanted or reaches the target area.

Optionally, it may be desirable to position a porous layer over the layer of therapeutic drug coated upon the medical device, in order to protect the therapeutic drug from releasing prematurely from the medical device, that is, prior to reaching its target tissue or circulation. Additionally, the porous layer may also be positioned over the layer of microparticles or nanoparticles encapsulating the therapeutic drug. If utilized, the porous layer is preferably biodegradable and slowly consumed during the insertion or deployment of the medical device, but can also be an inert stable layer. The thickness and type of material used to construct the porous layer is chosen based on the type of device, the insertion or deployment method used, and the length of time the device is in contact with body fluids prior to reaching its target tissue or circulation. Thus, various devices and applications require porous layers which degrade at different rates. However, most of the porous layer is preferably dissolved by the time the medical device reaches its target tissue or circulation in order for the therapeutic agent to be quickly and effectively released.

Alternatively, instead of a porous layer deposited over an existing layer of microparticles or nanoparticles, the material of these particles may be selected such that the biodegradation or bioerosion of the encapsulation material occurs at a rate which does not allow the therapeutic agent to be released prematurely.

The release profile of the drug from the microparticles or nanoparticles is determined by many factors including the drug solubility and the thickness and porosity of the microcapsules. The microcapsules of the invention may either be rupturable to release their contents or may be degradable such that they will open when left against the lumen walls. Thus, the particles or capsules may release their contents through diffusion or by rupturing due to the application of external forces. The particles or capsules may also be consumed by the phagocytic, chemotactic, and cytotoxic activities of surrounding cells. For example, macrophages are important killer T-cells and by means of antibody-dependent cell-mediated cytotoxicity (ADCC) they are able to kill or damage extracellular targets. Additionally, the drugs may be released by activating a trigger mechanism, or having it activated passively by the physiological conditions.

In one embodiment of the invention, the drug-coated medical device can be configured as at least one of, or any portion of, a catheter, an angioplasty device, a stent, a vascular or other graft, a cardiac pacemaker lead or lead tip, a cardiac defibrillator lead or lead tip, a heart valve, a suture, a needle, a guide wire, a cannula, a pacemaker, a coronary artery bypass graft (CABG), an abdominal aortic aneurysm device (Triple A device) or an orthopedic device, appliance, implant or replacement. In a further embodiment, the medical device can also be configured as a combination of portions of any of these devices. The drug may be coated on the entire surface of the medial device or a portion thereof. For example, the entire structure may be coated with a type of therapeutic agent, or only a specific portion, which will contact a target area, may be coated.

One example of a medical device useful in the present invention is a balloon catheter. In this embodiment, which requires a therapeutic drug to be delivered to an internal tissue site or circulation, the process and catheter is incorporated into a conventional percutaneous transluminal angioplasty (PTA). As well known in the art, a balloon catheter comprises a long, narrow hollow tube tipped with a miniature, deflated balloon, which is maneuvered through the cardiovascular system, and to an occlusion site. Once in the proper position, the balloon is inflated into contact with the lumen to be treated. The dilation catheter of the present invention may include any dilation catheter well known to those skilled in the art, to which therapeutic agents and controlled release carriers or particles are applied. “Particles” as the term is used herein includes liposomes, microparticles, nanoparticles and aggregates of the drug. The therapeutic drug is coated upon the balloon surface, which provides an adequate surface area to apply an effective amount of therapeutic agent. Any balloon catheter, whether capable of use in angioplasty or not, may be employed for local delivery of a therapeutic drug. Indeed, it is desirable that the balloon be elastic or have a high degree of elastic stress response.

An additional non-limiting example of a medical device useful in the present invention includes a stent for placement in a body lumen. As known in the art, stents are tubular support structures that are implanted inside tubular organs, blood vessels or other tubular body lumens. The stent is made of any suitable metallic (e.g. stainless steel, nitinol, tantalu, etc.), polymeric (e.g. polyethylene terephthalate, polyacetal, polylactic acid, polyethylene oxide-polybutylene terephthalate copolymer, etc.) or biodegradable material. Stents can have either solid walls or lattice like walls, and are usually either balloon expandable or self-expanding. Preferably, the stent is metallic and configured in a mesh design. A stent can be delivered on a catheter and expanded in place or allowed to expand in place against the vessel walls. The therapeutic drug of the present invention, which inhibits proliferative activity in the cardiovascular system, can be coated upon any stent of choice, chosen for optimal mechanical features. However, in-stent restenosis is preferably treated by using a drug-coated balloon catheter. Specifically, the drug-coated balloon catheter is used instead of an additional stent in order to treat the restenosis caused by the existing implanted stent.

In a preferred embodiment, a drug-coated or drug bound balloon catheter is utilized to release the therapeutic agents having anti-proliferative activity into the body tissue or circulation. However, utilizing a drug-coated stent is preferred when binding or coating the therapeutic agent to the metallic stent is advantageous over binding or coating the agent to the balloon material.

The therapeutic agent, preferably encapsulated in a particle or a controlled release carrier, or aggregated to a desirable/pre-selected size, for efficient uptake by a macrophage, is applied to the surface of the medical device by coating methods known in the art, including, but not limited to spraying, dipping, rolling, brushing, solvent bonding, adhesives or welding or by binding the microparticle or aggregates to the surface of the medical device by any chemical method known in the art. Furthermore, if the medical device has folds, corrugations, cusps, pores, apertures, or the like, the therapeutic agent or particle encapsulating the therapeutic agent may be embedded, i.e., mechanically trapped, within the medical device without the use of adhesives. In addition to the drug coated on the medical device, an additional dosage of the therapeutic drug, which inhibits proliferation in the cardiovascular system, may be applied by conventional delivery methods discussed above, (e.g., orally, intravenously) or may be injected through the medical device. For example, the therapeutic drug may be injected through the guiding catheter via the same method and procedure used to inject the contrast dye commonly used during a PTA. The particles are preferably selected from the group consisting of lipids, microparticles, nanoparticles, or the drug itself in aggregates, flocculates or the like.

The therapeutic drugs useful in the present invention preferably inhibit the proliferation of vascular smooth muscle cells. In one embodiment, the therapeutic drugs directly alter smooth muscle cell activity by altering cellular metabolism, inhibiting protein synthesis, or inhibiting microtubule and microfilament formation, thus affecting morphology. The therapeutic drug may also include inhibitors of extracellular matrix synthesis or secretion. Thus, in one embodiment, the methods and dosage forms of the present invention are useful for inhibiting vascular smooth muscle cells by employing a therapeutic agent that inhibits the activity of the cell, i.e. inhibits proliferation, contraction, migration or the like, but does not kill the cell. However, in a further embodiment, the methods and dosage forms of the present invention are useful for inhibiting target cell proliferation by employing a therapeutic agent that is cytotoxic to the cell.

The therapeutic agent, may directly or indirectly inhibit the activity of the smooth muscle cells, thus inhibiting or suppressing proliferation of the smooth muscle cells. For example, in one embodiment, the therapeutic agent may directly inhibit the cellular activity of the smooth muscle by inhibiting proliferation, migration, etc. of the smooth muscle cells. In a further embodiment, the therapeutic agent may inhibit the cellular activity of surrounding cells, whose activity initiates, assists or maintains proliferation of smooth muscle cells. Thus, smooth muscle cell proliferation is indirectly inhibited or suppressed by the inhibition or suppression of the metabolic activities of the surrounding cells, whose activities maintain smooth muscle cell proliferation.

In a preferred embodiment, the therapeutic drug encapsulated and coated on the medical device is used for reducing, delaying or eliminating restenosis following angioplasty. Reducing restenosis includes decreasing the thickening of the inner blood vessel lining, that results from stimulation of smooth muscle cell proliferation following angioplasty. Delaying restenosis includes delaying the time until onset of visible hyperplasia following angioplasty, and eliminating restenosis following angioplasty includes completely reducing and/or completely delaying hyperplasia to an extent which makes it no longer necessary to intervene. Methods of intervening include re-establishing a suitable blood flow through the vessel by methods such as, for example, repeat angioplasty and/or stent placement, or CABG.

One example of a group of drugs useful in the present invention to inhibit proliferative activity in the cardiovascular system, specifically smooth muscle cell proliferation, are bisphosphonates (BP). Bisphosphonates, formerly called diphosphonates, are compounds characterized by two C—P bonds. If the two bonds are located on the same carbon atom (P—C—P) they are termed geminal bisphosphonates. Bisphosphonates indirectly inhibit smooth muscle cell proliferation by metabolically altering surrounding cells, namely macrophages and/or monocytes. Bisphosphonates when encapsulated in liposomes or nanoparticles or aggregated in aggregates of a specific size, are taken-up, by way of phagocytosis, very efficiently by the macrophages and monocytes. Once inside the macrophages, the liposomes are destroyed and release the encapsulated bisphosphonates, which inhibit the activity of the macrophages. Since macrophages, in their normal state, are recruited to the areas traumatized by angioplasty or other intrusive intervention and initiate the proliferation of smooth-muscle cells (SMC), inhibiting the macrophages' activity will inhibit the proliferation of SMC. Once released and taken-up by the macrophages, the bisphosphonates will have a sustained anti-proliferative activity for the lifetime of the macrophages. Thus, prolonged release of the bisphosphonates is not required in order to sustain inhibition. Representative examples of bisphophonates suitable for use in the present invention are alendronate, clodronate, and pamidronate.

In a preferred embodiment of the present invention, the therapeutic drug is encapsulated in relatively large liposomes that are preferably taken up by cells such as monocytes and macrophages. The structure and composition of the liposomes are discussed supra. Additionally, the liposomes may be greater than 100 nanometers in size and contain, for example, a bisphosphonate drug.

In one embodiment, the drug, such as, for example, a bisphosphonate may be encapsulated in a liposome and coated upon a suitable medical device. Coating methods and suitable medical devices are discussed supra. For example, the liposomal bisphosphonates may be coated on a balloon catheter and suspended in a macrostructure such as glucose or gelatin, or chemically bound to the surface. Thereafter, the balloon catheter is effectively maneuvered through the cardiovascular system and to an occlusive site. Once in the proper position, the balloon is inflated into contact with the lumen to be treated. The liposomes, which encapsulate the bisphosphonate therapeutic drugs, are then released from the medical device and are present in the tissue and in the circulation, ready for uptake by macrophages, locally and systemically.

Upon the release of the liposomes into the lumen of the affected area and immediate uptake by the macrophages, restenosis is inhibited. For example, bisphosphonates may prevent monocytes from developing into macrophages by altering their cellular metabolism. Furthermore, the BP may also inhibit cellular activity of macrophages thereby altering their biological function as the central effector and regulatory cell of the inflammatory response. Therefore, while macrophages are recruited to the traumatized area, these cells can not initiate the inflammatory process that turns into restenosis. The release of the Liposomal BP (LBP) can be carried out systemically and/or locally, and is taken-up by macrophages systemically and locally.

In a further embodiment, the medical device may also carry therapeutic agents, such as, for example, anti-spasmodic, anti-thrombogenic, and anti-platelet agents, antibiotics, steroids, and the like, in conjunction with the anti-proliferative agent, to provide local administration of additional medication.

It is to be understood that the embodiments and variations shown and described herein are merely illustrative of the principles of the present invention. Therefore, various adaptations and modifications may be implemented by those skilled in the art without departing from the spirit and scope of the present invention.

We claim:

1. A method of delivering a drug having anti-proliferative activity in the cardiovascular system to a tissue or circulation, comprising:
contacting the tissue or circulation with a device which is coated with the drug; and
releasing the drug into the circulation or the tissues surrounding the device in less than 5 minutes after the contacting step.

2. The method according to claim 1, wherein the drug is a medicine.

3. The method according to claim 2, wherein the drug is a bisphosphonate.

4. The method according to claim 3, wherein the drug is selected from the group consisting of alendronate, pamidronate and clodronate.

5. The method according to claim 1, wherein the drug is encapsulated in a particle.

6. The method according to claim 1 wherein the drug is aggregated to form aggregates of a pre-selected size.

7. The method according to claim 5, wherein the particle is of a size taken-up by target cells of the white blood-cell lineage.

8. The method according to claim 7, wherein the target cells are selected from the group consisting of monocytes and macrophages.

9. The method according to claim 1, wherein the device is configured as at least one of, or any portion of, a catheter, an angioplasty device, a stent, a vascular or other graft, a cardiac pacemaker lead or lead tip, a cardiac defibrillator lead or lead tip, a heart valve, a suture, a needle, a wire guide, a cannula, a pacemaker, a CABG, a Triple A device, or an orthopedic device, appliance, implant or replacement.

10. A method of delivering a drug having anti-proliferative activity in the cardiovascular system to a tissue or circulation, comprising:
contacting the tissue or circulation with a device which is coated with the drug, wherein the drug is quickly released into and immediately absorbed by the circulation or the tissues surrounding the device in a short time after the contacting step and wherein the drug is encapsulated in a particle.

11. The method according to claim 10, wherein the particle is an inert polymeric particle.

12. The method according to claim 10, wherein the particle is a microparticle.

13. The method according to claim 10, wherein the particle is a nanoparticle.

14. The method according to claim 10, wherein the particle is an aggregate of the drug molecules.

15. The method according to claim 10, wherein the particle is a controlled release carrier.

16. The method according to claim 15, wherein the controlled release carrier is a liposome.

17. The method according to claim 16, wherein the liposome is greater than 100 nm in size.

18. The method according to claim 10, wherein the particle is of a size taken-up by target cells of the white blood-cell lineage.

19. The method according to claim 18, wherein the target cells are selected from the group consisting of monocytes and macrophage.

20. The method according to claim 1 or 10, wherein the drug is released within 1 minute of initial contact with the tissue or circulation.

21. The method according to claim 1 or 10, wherein the drug is released within 30 seconds of initial contact with the tissue or circulation.

22. The method according to claim 1 or 10, wherein the drug has sustained activity for inhibiting proliferation of smooth muscle cells.

23. A medical device comprising:
a layer of a therapeutic drug applied on the exterior of the medical device, the therapeutic drug having anti-proliferative activity in the cardiovascular system, wherein the medical device is contacted with a tissue or circulation such that the drug is released from the medial device and into the surrounding tissue or circulation in less than 5 minutes after the contacting step.

24. A medical device comprising:
a plurality of particles dispersed on the surface of the medical device, each particle comprising a therapeutic drug or a combination of therapeutic drugs having anti-proliferative activity in the cardiovascular system, wherein the particles are selected from the group consisting of liposomes, microparticles, nanoparticles, and drug aggregates, and wherein the medical device is contacted with a tissue or circulation such that the drug is released from the particle and into the surrounding tissue or circulation in less than 5 minutes after the contacting step.

25. A medical device comprising:
a plurality of particles, which are supported within the matrix of a macrostructure, dispersed on the surface of the medical device, each particle comprising a therapeutic drug or a combination of therapeutic drugs having anti-proliferative activity in the cardiovascular system, wherein the particles are selected from the group consisting of liposomes, microparticles, nanoparticles, and drug aggregates, and wherein the medical device is contacted with a tissue or circulation such that the drug is released from the particle, and into the surrounding tissue or circulation in less than 5 minutes after the contacting step.

26. The medical device of claim 23, wherein the drug is absorbed into the surrounding tissue or circulation upon the release from the medical device.

27. The medical device of claims 24 or 25, wherein the drug is absorbed into the surrounding tissue or circulation upon the release from the particle.

28. The medical device of claim 23, 24 or 25, wherein the drug inhibits the proliferation of smooth muscle cells.

29. The medical device of claim 23, 24 or 25, wherein said therapeutic drug is a bisphosphonate.

30. The medial device of claim 29, wherein the drug is selected from the group consisting of alendronate and clodronate.

31. The medical device of claim 25, wherein the macrostructure is selected from the group consisting of fibrin gels, hydrogels and glucose.

32. The medical device of claim 24, wherein the particles are supported within the matrix of a macrostructure.

33. The medical device of claim 23, wherein the therapeutic drug is applied to the surface of the medical device by coating methods selected from the group consisting of spraying, dipping, rolling, brushing, and solvent bonding.

34. The medical device of claim 24 or 25, wherein the particles are attached to the medical device surface by coating methods selected from the group consisting of spraying, dipping, rolling, brushing, solvent bonding, adhesives and welding.

35. The medical device of claim 24 or 25, wherein the particles are mechanically trapped on the surface or within the medical device.

36. A method for forming a medical device capable of delivering therapeutic drugs to a tissue or circulation comprising the steps of:
obtaining a suitable medical device which will contact the tissue or circulation, such that said therapeutic drugs are released; and
applying a layer of therapeutic drugs to the surface of the medical device.

37. A method for forming a medical device capable of delivering therapeutic drugs to a tissue or circulation comprising the steps of:
obtaining a suitable medical device to contact the tissue or circulation, such that said therapeutic drugs are released; and
applying a plurality of drug-containing particles to the surface of said medical device, wherein the particles are selected from the group consisting of liposomes, microparticles nanoparticles, and drug aggregates.

38. A method of treating smooth muscle cell proliferation comprising the steps of:
providing a medical device having a plurality of particles on the exterior of the balloon, each of the particles comprising at least one therapeutic drug having anti-proliferative activity, wherein the particles are selected from the group consisting of liposomes, microparticles, nanoparticles and drug aggregates;
contacting said medical device with the tissue or circulation to be treated such that the therapeutic drug is quickly released from the particles and is effective immediately.
